(19) United States
(12) Patent Application Publication
(10) Pub. No.: US 2007/0026064 A1
(43) Pub. Date: Feb. 1, 2007

(54) Pharmaceutical dosage forms having watermark-type identification and authentication inditia

(76) Inventors: Steven L. Yoder, Skippack, PA (US); Brian D. Korchok, Schwenksville, PA (US); Frederick R. Kettinger, Norristown, PA (US)

Correspondence Address: LUCAS & MERCANTI, LLP, 475 PARK AVENUE SOUTH, 15TH FLOOR, NEW YORK, NY, 10016, US

(21) Appl. No.: 11/495,378

(22) Filed: Jul. 28, 2006

Related U.S. Application Data
(60) Provisional application No. 60/704,206, filed on Jul. 29, 2005.

(51) Int. Cl. A61K 9/28; A61K 9/20; B05D 3/02

(52) U.S. Cl. 424/464; 427/2.14

(57) ABSTRACT

Oral solid dosage forms having holographic markings or identification marks such as a watermark thereon and methods of preparing the same are disclosed. One preferred method of applying an identification mark on a pharmaceutical dosage form described herein includes applying a film coating having a first color to an oral solid dosage form such as a tabet and thereafter applying an identification mark to the film coated oral solid dosage form using an ink which not only contains a pearlescent pigment therein but also has a second color which is substantially similar in color to the first color used for film coating.

### CROSS-REFERENCE TO RELATED APPLICATIONS

This application claims the benefit of priority from U.S. Provisional Application Ser. No. 60/704,206, filed Jul. 29, 2005, the contents of which are incorporated herein by reference.

### FIELD OF THE INVENTION

This invention relates to pharmaceutical oral dosage forms such as tablets having unique identification markings similar to a watermark thereon. The invention also relates to methods of applying the markings to tablets and the like as well as methods of reducing the counterfeiting of pharmaceuticals.

### BACKGROUND OF THE INVENTION

In recent years there has been a need for increased control and tracking of pharmaceutical dosage forms. There have been suggestions to implement various tracking devices such as bar codes and holograms, etc. on the bulk packages and unit dosage packs sent to pharmacies from manufacturers. Others have suggested employing bar codes on oral dosage forms as a way of increasing the tracking of the tablets. If the tablets could be scanned before being given to the patient by the healthcare provider, it is believed that there would be a significant reduction in medication administration errors in hospitals and pharmacies. Further information regarding the efforts in this regard are found, for example, in U.S. Pat. Nos. 5,942,444, 5,992,742 and 6,543,692.

Recently, there has been increased interest in reducing and, if possible, eliminating counterfeit pharmaceuticals. This need is particularly acute in the area of tablets and oral solid dosage forms. The manufacturer's need for security and tracking of their pharmaceuticals is at odds in certain respects with the commercial appeal of the products. Patient acceptance of a product is highly dependent on the visual appearance and elegance of pharmaceutical tablet. Therefore, any brand identity markings or anti-counterfeit measures imprinted on a tablet should be either invisible to the consumer or otherwise not detract from the appearance of the dosage form. The present invention addresses this need.

### SUMMARY OF THE INVENTION

In one aspect of the invention there are provided methods of applying an identification mark on a pharmaceutical dosage form. The methods are preferably used to provide a manufacturer with the means for uniquely identifying the tablets and reducing the chances for counterfeiting of the product by other parties. Thus, the methods generally include:

a) providing an oral solid dosage form;

b) applying a film coating having a first color to the oral solid dosage form; and

c) applying an identification mark to the film coated oral solid dosage prepared as a result of step b), using an ink containing a pearlescent pigment therein.

The ink used to apply the identification mark to the film coated tablet has a second color which is substantially similar in color to the first color.

In an alternative aspect of the invention, the pearlescent pigment is employed in the film coating rather than in the ink. Specifically, the methods for applying an identification mark on a pharmaceutical dosage form include:

a) providing an oral solid dosage form;

b) applying a film coating containing a pearlescent pigment therein and having a first color to said oral solid dosage form;

c) applying an identification mark to the film coated oral solid dosage prepared as a result of step b), using an ink having a second color which is substantially similar in color to said first color.

Further aspects of the invention include tablets made by the methods described herein.

In preferred aspects of this invention, the identification markings are applied to the oral solid dosage form using a roto-gravure printer, a pad printer or a high speed ink jet printer.

As a result of the present invention, there is provided a technology that allows a pharmaceutical company to distinguish among tablets and other dosage forms. The techniques enhance brand value for the manufacturer and make the tablets difficult to counterfeit. The unique label markers on the tablets are non-obtrusive yet can help reduce medication errors and to enhance patient compliance. In those aspects of the invention where a bar code or micro bar code or a microscopic taggant are used, the integrity of pharmacy supplies is increased since the individual tablets rather than the bulk containers can be scanned or otherwise inspected for tablet integrity and accuracy. The efforts of counterfeiters can also be thwarted by this identification system which provides unique printed images since the identification marks applied to the tablets are not easily copied and include more than just color, shape or word markings on tablets.

In particularly preferred aspects of the invention, the artisan uses a substrate with similar color characteristics, such as by film coating or colorant in the compressible tablet formulation or a combination of both, as that included in the ink used for the watermarking solution containing the pearlescent pigment. This enables one to produce a holographic-like image onto the tablet or substrate. Such images can usually only be seen clearly if the light is reflected off of the tablet or substrate at specific viewing angles.

For purposes of the present invention, “similar color” and “substantially similar” in color when used in connection with the film coatings and inks shall be understood by those of ordinary skill to mean a combination of colors which when measured with a color spectrophotometer, are plotted in the same region of the three-dimensional (L,a,b) colorspace. Stated another way, the combination of colors allow a holographic effect for the marking on the tablets when viewed with the eye in a visible light environment. Substantially similar in color shall further be understood to include colors which are closely related in the visible spectrum, yet have discernable differences which are visible to the eye or measuring apparatus. Similarly, “lighter” and “darker” shall be understood to have their generally accepted dictionary definitions and provide for a relative demarcation as between two or more colors which can be measured either by the eye or with a suitable apparatus.

An identification mark for purposes of the present invention shall include any symbol, marking, logo, indicia, etc. which can be used as a source indicator for the tablets.

For purposes of the present invention, it will be understood that the tablets or other oral solid dosage forms used herein will have sufficiently low friability so as to be able to undergo the processing steps and manipulations required to apply the markings described herein without causing significant breakage of the dosage forms.

For purposes of the present invention, “substantially translucent” shall be understood to mean almost transparent; allowing light to pass through somewhat diffusely but nonetheless allowing the object covered therewith to be seen there through with sufficient clarity so as to be identifiable.

For purposes of the present invention, the terms “readable” and “detectable” as used in conjunction with the markings applied to the dosage forms described herein shall be understood to embrace all such markings which can be recognized by optical apparatus, i.e. bar code reader, etc. or other detection devices such as HPLC, etc.

### BRIEF DESCRIPTION OF THE DRAWINGS

FIG. 1 is a photograph showing compressed tablets prepared in accordance with Example 13 of the present invention.

FIGS. 2-5 are photographs showing compressed tablets containing watermark indicia and coatings which were prepared in accordance with Examples 1-12

### DETAILED DESCRIPTION OF THE INVENTION

The first preferred aspect of the invention includes a method of applying an identification mark on a pharmaceutical dosage form. It comprises the steps of:

a) providing an oral solid dosage form;

b) applying a film coating having a first color to said oral solid dosage form; and

c) applying an identification mark to the film coated oral solid dosage prepared as a result of step b), using an ink containing a pearlescent pigment therein, said ink having a second color which is substantially similar in color to said first color.

An alternative thereto provides another method of applying an identification mark on a pharmaceutical dosage form. It comprises the steps of:

a) providing an oral solid dosage form;

b) applying a film coating containing a pearlescent pigment therein and having a first color to said oral solid dosage form; and

c) applying an identification mark to the film coated oral solid dosage prepared as a result of step b), using an ink having a second color which is substantially similar in color to said first color.

Although the visual effects achieved by either method are similar, the invention is described with reference to the first embodiment. The method of the invention can provide the identification mark onto a film coated oral solid dosage form so that it appears substantially translucently or clearly visible on the oral solid dosage form. Alternatively, the methods of the invention provide the identification mark onto the film coated oral solid dosage form so that it is substantially invisible when viewed in the spectrum of visible light or even only in ultraviolet or infrared light. In other words, depending upon the ink selected for applying the mark to the tablet, the identification mark can be readable with detection equipment which does not depend upon visible light waves. A further aspect of the invention is for the identification mark to readable with a bar code scanner. When this option is desired, the identification marker can be a bar code or a micro bar code.

The methods of the present invention can be employed on any number of oral solid dosage forms including tablets, caplets, etc. Preferably, the oral solid dosage form is a compressed tablet. It will be understood by those of ordinary skill that even though the invention is described at times in terms of a step of “providing an oral solid dosage form”, the invention also includes embodiments where the processes are carried out on tablets which are merely present in an area to be coated so that the recited step a) of “providing” is not specifically required and the inventive method begins with applying a coating to tablets or other oral solid dosage forms.

The pharmaceutical dosage forms are preferably film coated prior to the application of the ink-based identification marking. While a wide variety of tablet film coatings can be employed in carrying out this part of the invention, preferred film coatings are capable of adhering to both the tablet core, which is typically a microcrystalline cellulose-based compressed tablet, and the ink-based marking. It is preferably based on an edible film coating composition well known to those of ordinary skill. A non-limiting list of suitable film coatings include those available from Colorcon under the OPADRY® and OPAGLOS® brand names. Generally, however, such suitable film coatings well known to those of ordinary skill are based on a blend of a cellulosic polymer or polyvinyl alcohol (PVA), plasticizer and other film coating ingredients. The film coating may also be free of pigment or include a sufficient amount of pigment which imparts added luster or otherwise enhances the pearlescent marking applied to the oral dosage form.

The amount of film coating applied to the core before the marking is applied will depend upon the needs of the artisan, the type of marking applied and other parameters know to those of ordinary skill. Generally, the tablet cores are coated to a weight gain of from about 0.25 to about 15% by weight. Preferably, the film coatings are applied to weight gains of from about 1 to about 10% by weight. The film coating can also comprise a series of separate film coatings, each of which contributing to the weight gain ranges mentioned herein.

After the tablets are film coated, the marking of the tablets with a pearlescent pigment can be carried out. The pearlescent pigments can be incorporated into most pharmaceutically acceptable inks. Some preferred inks include the OPACODE® brand inks available from Colorcon of West Point, Pa. Other inks will be apparent to those of ordinary skill. In any case, the inks are prepared in standard fashion except that a portion or all of the pigment is replaced with a pearlescent pigment. Preferred inks are those which contain from about 0.25 to about 75% by weight pearlescent pigments therein. More preferred inks are those which contain from about 1 to about 30% by weight pearlescent pigments therein. Furthermore, in certain aspects of the invention where a holographic effect is desired, it is preferred that the ink contain at least about 5% or greater pearlescent pigment. In those aspects where watermark-type features are desired, lesser amount of the pearlescent pigment in the ink can be used.

As will be appreciated, there are several types of commercially available pearlescent pigments. It is preferred that the pearlescent pigment is micaceous. The pearlescent pigment can be a mica coated with titanium dioxide, iron oxide, etc. and combinations thereof.

In one preferred embodiment of the invention, the pearlescent pigments included are based on titanium dioxide platelets, also known as platy TiO₂, such as those available from Engelhard and/or those described in U.S. Pat. No. 5,611,851, the disclosure of which is incorporated herein by reference. Such products can be referred to as platelets of titanium dioxide. A non-limiting list of suitable pearlescent platy TiO₂ pigments include green, blue, violet, red, gold, orange, and pearl. See also, for example, Greenstein, L. M. “Nacreous Pigments” Encyclopedia of Polymer Science and Technology, Vol. 10, pp 193-215. Such pigments are available in a wide variety of colors such as reds, golds, violets, greens, etc.

In an alternative aspect of this embodiment, the pearlescent pigment is a micaceous pearlescent pigment such as those available under the tradename Candurin from Merck as mentioned above. See also PCT publication number WO 00/03609, the disclosure of which is incorporated herein by reference. A non-limiting list of suitable pearlescent pigment products include Candurin silver fine, silver sheen, silver luster and sparkle silvers, etc. various “sugar” products like banana sugar or others having a white color and gold, red or blue highlights. Still others include those having various colors, e.g. reds, bronzes, coppers having glitter or luster finishes. Other suitable micaceous pigments are those containing mica coated with Titanium dioxide, ferrous oxide, etc. and combinations thereof. The only limitation on the pearlescent pigments included in the powders and other formulations described herein is that they must be capable of being substantially homogeneously combined with the other ingredients and they must be capable of providing a high pearlescent finished coating on the coated, edible article without substantially negatively effecting the organoleptic qualities of the finished product.

Regardless of the source of the pearlescent pigment for the ink or film coating, it is incorporated into the ink or film coating by replacing all or a portion of the pigment typically employed in the formulation.

Rotogravure printing is one preferred embodiment in the method for applying the identification marks according to the present invention. Gravure processes begin with the engraving of the desired pattern or image into a plate, or about a roller. The use of a roller provides for a continuous process, printing the image repeatedly onto a moving web. Thus, in the continuous process, deemed a rotogravure process, the print image desired is carved into the surface of the roller, sometimes called a print or engraved cylinder. The printing ink dispersion or solution is provided in a trough. The rotating cylinder is mounted horizontally such that, a full height of the cylinder extends into the print solution in the trough. As the cylinder turns, it is flooded with print solution. A doctor blade, extending the height of the cylinder removes the excess dispersion solutions, leaving the dispersion in the carved image. The cylinder then turns to a nip with an impression roller, and prints onto a web moving through the nip. Thus, an image is placed on the tablet.

The invention also includes various other printing processes, including, preferably pad printing processes or ink jet printing processes including high speed ink jet printing.

In one preferred embodiment, the tablets are pad printed using an apparatus such as that described in PCT published application WO01/30573 A1, the contents of which are incorporated herein by reference. Such devices are capable of accurately and uniformly applying printing symbols and the like using a pad printer onto compressed tablets which are oriented on a conveyor unit at relatively high speed. The apparatus are also available from Printing International of Belgium.

As an alternative to the pad printing, ink jet printing of the markers onto the tablets can be employed. In such aspects of the invention, an ink jet printer replaces the pad printing device and the tablets are conveyed to the printer for marking(s). The ink jet printer allows for further identification of the tablets such as serialization (numbering of the tablets consecutively, etc.) without the need to stop the continuous printing process to change the printing pad or other marking device. Such modifications are said to be made in real time and allow a myriad of changes to be made. One such ink jet printer is available from Domino of Cambridge, UK. Alternatives include piezojet inkjet systems such those available from Xaar Technology Limited of Cambridge, UK or any other devices capable of delivering high resolution images onto oral solid dosage forms. Regardless of the printing apparatus employed, the ink selected for marking the tablets must be ingestible and meet all regulatory requirements for use in the pharmaceutical industry.

As mentioned above, the first color found in the film coating applied to the oral solid dosage form is substantially similar to that employed in the ink. This does not mean that they are exact matches. It only means that when one is applied to the surface containing the other, a holographic effect is observed when the finished product is observed. Thus, the color of the film coating can be darker than the second color used in the ink used to apply the mark to the oral solid dosage form after film coating. Alternatively; the first color found in the film coating applied to the oral solid dosage form can be lighter than the second color in the ink applied to the oral solid dosage form after film coating. While applicants are not bound by theory, it is believed that in certain preferred embodiments that certain visible improvements are more pronounced when ink applied to the film coated tablet is at least about as dark as the film coating. Some more particularly preferred aspects of the invention are achieved when the pearlescent containing inks are darker than the film coatings applied to the oral solid dosage forms.

The invention includes the tablets or oral solid dosage forms made by the process. Once the marking such as a bar code or logo has been applied, the invention also allows for the ready visual identification of the drug product via its uniquely colored label.

The invention further includes the ability to electronically scan a tablet to readily identify the tablet and or drug using the pearlescent marking. Such techniques for bar code reading are known to those of ordinary skill, see, for example U.S. Pat. Nos. 5,992,742 and 6,543,692, the disclosure of each of which is incorporated herein by reference. The invention includes the stand alone identification of the drug through visual means, the stand alone identification of the drug through electronic scanning means and or the combination of identification through visual and electronic means.

Within the preferred bar code marking aspects of the invention there are included the use of 2d data matrix bar codes and micro bar codes. Data Matrix is a matrix-type of 2D bar code that comes in a number of different array sizes and is identifiable by a finder pattern that occupies the perimeter of the optical code. The bottom and left edges of the code are solid black bars, forming an L-shaped pattern. The top and right edges of the code are made up of alternating black and white cells, allowing the reader to determine the number of rows and columns present in the matrix. The Data Matrix standard (ref) defines different types and levels of error correction, the preferred of which is ECC200. ECC200 uses a Reed-Solomon error correction scheme to allow the user to detect and correct a number of damaged bytes. Although most Data Matrix codes have an equal number of rows and columns, the standard allows for rectangular codes where there are twice the number of columns as rows.

Some 2D matrix codes can take on a number of allowed sizes or information densities. These types of codes must include easily detectable information that allows the reader to determine the bar code element size: the minimum height and width of a black or white area or “cell” in the code. Matrix codes typically possess a physical symmetry between the external size and the size of an individual cell. It is possible to determine the number of rows and columns of the matrix by knowing only the length of one side and the size of a single cell. For example, if an object within an image is found to be 100,000 pixels in height and the minimum distance between dark to light transitions is 7,962 pixels, the matrix height in elements can be calculated by dividing 100,000 by 7,962=13 elements high. For codes where only one physical size and information density is allowed, the number of rows and columns are known quantities. Although 2D bar codes are more compact than 1D codes, special care must be taken to ensure that the data stored in the code can be properly extracted. 1D codes provide “information redundancy” in the vertical direction. The taller the 1D codes, the greater the likelihood that a scan line will find and traverse an undamaged portion of the code thereby allowing a successful decode. 2D codes have built-in error detection and “error correction” data to allow the decoder to successfully decode a symbol even if part of the code is damaged or missing.

A micro barcode is about one-tenth the size of a standard barcode. The micro barcode was developed by Neorex, which is a Nagoya, Japan-based company. The scanner for reading the micro barcode has a specially developed lens that reads the micro barcode even if the lines are blurred. Any of the bar codes can be applied using the printing apparatus described herein, especially with the ink jet printers.

The invention includes the use of visible and or invisible pigments, colors and or inks to enable identification by light sources and scanning systems that do not rely upon visible light waves. A wide variety of suitable pigments and colorants are available from Colorcon, West Point, Pa. as well as other art recognized suppliers of pharmaceutically acceptable ingredients.

The invention includes the use of covert markers in and/or on tablets, in and/or on film coatings and in or on inks or other pigment systems either visible or invisible to the human eye when applied to solid dose forms. The authentication systems included in the systems of the present invention are those which globally approved or approvable covert markers which can be employed without materially effecting the active ingredient included as part of the dosage form.

The marking techniques of the present invention can also used in combination with covert markers. Covert markers contemplated for use herein include those available from Biocode of Bethlehem, PA and described, for example in U.S. Pat. No. 5,942,444, the disclosure of which is incorporated herein by reference. The amount of the covert marking included will depend upon several factors including the specific marking selected, the tablets being treated, etc. For example, the covert marking can be included as part of the film coating or ink used for the marking of the tablets. Preferably, however, the covert marking is applied to the tablet as part of the film coating with amounts of up to about 5 parts per million (ppm)/tablet. The exact amount employed can also vary somewhat based on the individual marked selected and the needs of the artisan. Thus, in some embodiments of the invention there may be markers present in amounts beyond that mentioned above.

These covert markers cannot usually be detected by the human senses, and extremely difficult if not impossible to detect through normal analytical techniques. The markers can be incorporated into a solid dosage film coating to enshroud the solid dose form thereby providing a continuous security “package” in the form of a coating around the solid dose form. The covert marking system thus provides information amounting to a “chemical bar code” to render data such as source of manufacture, date of expiration, channel of distribution, origin, etc. Hence, the covert marker can serve as a carrier of information that extends beyond the NDC code which is contained in the 2D bar code on the surface of the film coated tablet or other solid dose form. Analysis of the covertly marked tablet can be done using the Biocode lateral flow device (LFD) to provide a quick qualitative visual confirmation of the presence of the marker. In use, the surface of the tablet suspected of containing the covert marking is wetted with a small amount of water and a portion of the run off is processed with the LFD to confirm the presence of the covert marker. If desired, additional confirmation of the presence of the marker can be had using other chromatographic methods including HPLC or GC, etc.

The covert markers can also be used to identify the film coating, and/or ink and/or other pigmented systems applied to or contained within the tablet as well as other excipients in the tablet and/or the drug/active ingredient contained within or on the solid dosage form. As will be apparent to those of ordinary skill, other suitable taggants can be employed as either part of the ink marking formulation, film coating(s) or both.

### EXAMPLES

The following examples serve to provide further appreciation of the invention but are not meant in any way to restrict the effective scope of the invention.

Manufacture of Slugs Used for Formula Evaluation

A placebo blend was prepared with the following composition: 48.625% microcrystalline cellulose, 48.625% Pregelatinized Corn Starch, 2% Stearic Acid, 0.5% Colloidal silicon dioxide (Cab-O-Sil), and 0.25% Magnesium Stearate. Five grams of the placebo blend was placed into a flat faced 1⅛″ diameter die and compressed using a Carver Hydraulic press (Model C) for 15 seconds at 20,000 lbs force. Finished slugs were 1⅛″ diameter by ¼″ thick and were white.

### Examples 1-12

In these examples, standard manufacture processes were used to formulate the inks. In each case, an Opacode base formulation prepared as disclosed in commonly assigned U.S. Pat. No. 5,006,362, the disclosure of which is incorporated herein by reference. The inks were made to incorporate four different amounts by weight of gold pearlescent pigment into the various solutions. Once the inks were fully into solution, they were tested utilizing standard test methods and there were no adverse affects noted.

Standard concave round placebo tablets were prepared by coating them with one of three Opadry film coatings (Colorcon, West Point, Pa.) to about a 3% weight gain utilizing standard coating parameters. Specifically, a charge containing a quantity of placebos was spray coated with the Opadry in an O'Hara LabCoat I fully perforated side-vented coating pan equipped with a 12″ insert and 1-Spraying Systems JAU gun (¼ JAU-SS, 60/100SS nozzle and 134 255-45-SS air cap).

The average coating parameters are: inlet temperature (IT) 63° C., exhaust temperature (ET) 43° C., coating bed temperature (BT) 43° C., airflow 120-130 cfm, air pressure −0.1 in. of water, fluid delivery rate (FDR) 8 g/min, Atomizing air pressure (AP) 35 psi, pan speed (PS) 18 rpm. A theoretical coating weight gain of 3.0% was applied to the tablets. An acceptable coating was achieved and the tablets were removed from the vented coating pan. This procedure was done three times to provide red, orange and green coated tablets. See Table below:

| | 7% | 10% | 15% | 20% |
|---|---|---|---|---|
| | Pearlescent | Pearlescent | Pearlescent | Pearlescent |
| | Pigment | Pigment | Pigment | Pigment |
| Color | Example # | Example # | Example # | Example # |
| Red | 1 | 2 | 3 | 4 |
| Orange | 5 | 6 | 7 | 8 |
| Green | 9 | 10 | 11 | 12 |

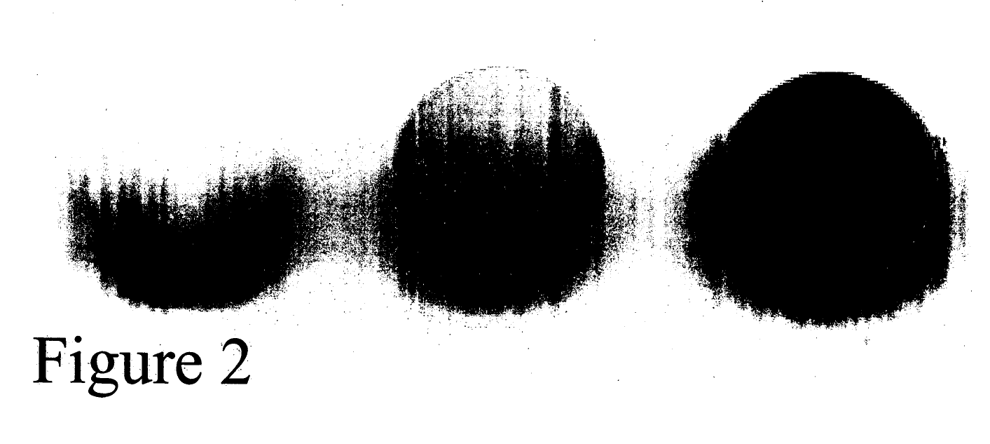

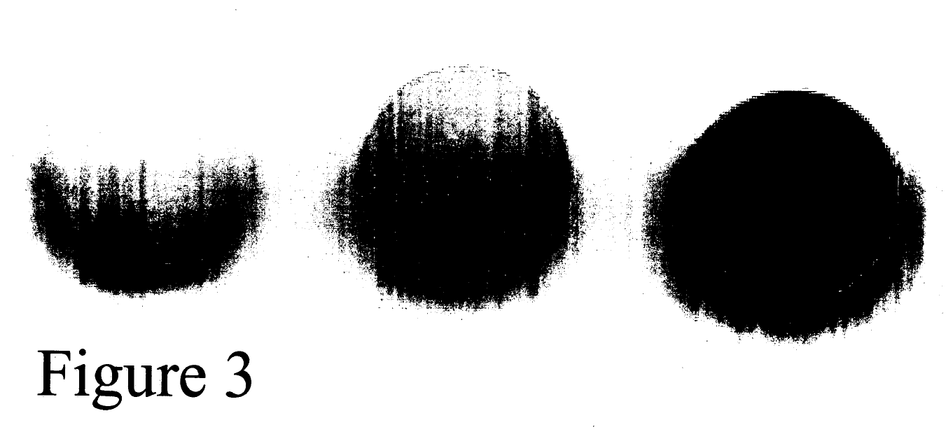

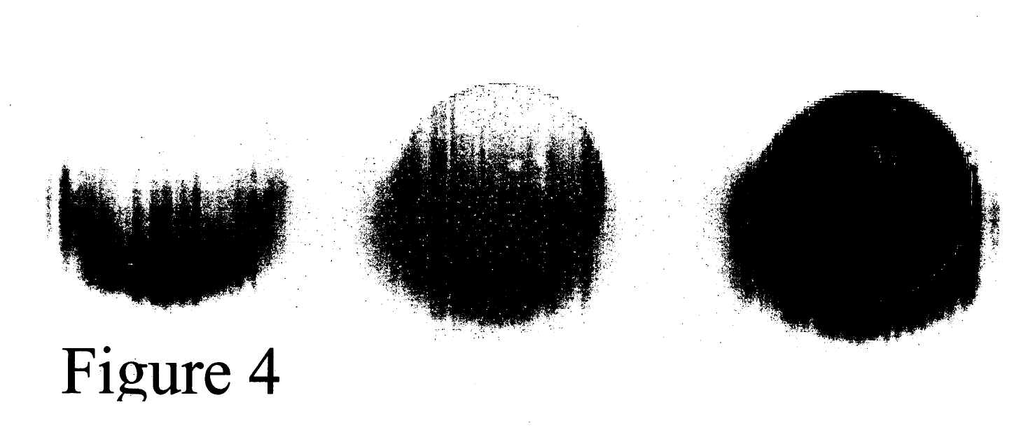

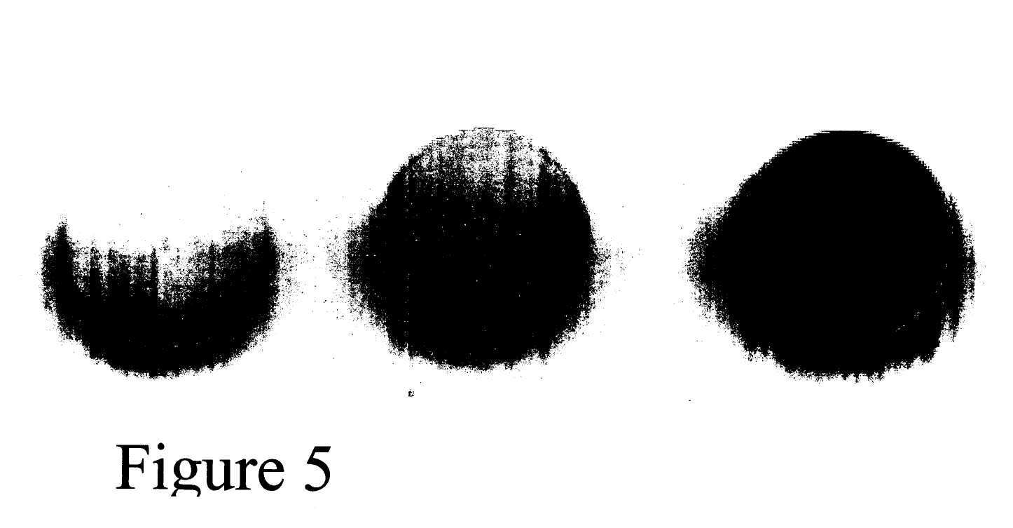

Once the tablets were coated we set-up the Hartnett Roto-Gravure printer. The pearlescent-pigment containing ink was poured into the ink well. The etch roller was equipped with an etching of the Colorcon logo and the tablets were loaded onto belt of the printing press. The press was set for a standard speed and the belt was engaged. The tablets began to orient themselves enabling the ink to transfer from the transfer rollers to the tablets. The ink dried in an adequate amount of time and the tablets could be discharged from the machine immediately. There was good adhesion and no imperfections were noticed. Several runs were made so that each color film coated tablet was test with each of the different inks made with the differing amounts of pearlescent inks. Representative samples of the work are shown in FIGS. 2-5. It will be appreciated that the holographic nature of the imprint is somewhat diminished by the fixed position of the tablets in the picture. The pearlescent effect of the imprint was visible, especially in the orange and green tablets where the interaction with gold pearlescent logo provided the holographic effect when the tablets were tilted in the light.

### Example 13

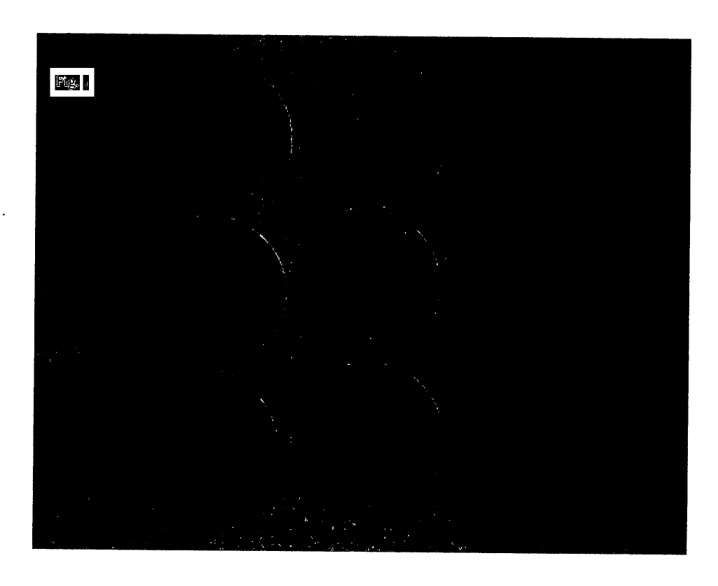

In this example the procedure mentioned above was repeated using tablets film coated with a gold color before the 15% pearlescent pigment gold ink was applied. See FIG. 1 for the resulting tablets which demonstrate the holographic effect when the color of the tablet is substantially similar to the color or the ink.

### Examples 14-25

In these examples, the procedure used in Examples 1-12 is repeated except that the pearlescent pigment is contained in the film coating Opadry fX (Colorcon, West Point, Pa.) and the logo is applied using a non-pearlescent Opacode ink.

What is claimed is:

1. A method of applying an identification mark on a pharmaceutical dosage form, comprising:
a) providing an oral solid dosage form;
b) applying a film coating having a first color to said oral solid dosage form; and
c) applying an identification mark to the film coated oral solid dosage form prepared as a result of step b), using an ink containing a pearlescent pigment therein, said ink having a second color which is substantially similar in color to said first color.

2. The method of claim 1, wherein the identification mark applied to film coated oral solid dosage form appears substantially translucently on said oral solid dosage form.

3. The method of claim 1, wherein the identification mark applied to film coated oral solid dosage form is substantially invisible when viewed in the spectrum of visible light.

4. The method of claim 1, wherein identification mark is visible in ultraviolet light.

5. The method of claim 1, wherein the identification mark is readable with a bar code scanner.

6. The method of claim 1, wherein the identification mark is readable with detection equipment which does not depend upon visible light waves.

7. The method of claim 1, wherein the identification marker is a bar code.

8. The method of claim 1, wherein the identification marker is a micro bar code.

9. The method of claim 1, wherein said oral solid dosage form is a compressed tablet.

10. The method of claim 1, wherein said applying of the identification mark is carried out with a high speed ink jet printer.

11. The method of claim 1, wherein said applying of the identification mark is carried out with a rotogravure printer or pad printer.

12. The method of claim 1, wherein the ink contains from about 0.25 to about 75% by weight pearlescent pigments therein.

13. The method of claim 12, wherein the ink contains from about 1 to about 30% by weight pearlesent pigments therein.

14. The method of claim 1, wherein said pearlescent pigment is a micaceous pearlescent pigment.

15. The method of claim 1, wherein said pearlescent pigment is selected from the group consisting of mica coated with a member of the group consisting of titanium dioxide, iron oxide and combinations thereof.

16. The powder mixture of claim 1, wherein said pearlescent pigment comprises titanium dioxide platelets.

17. The method of claim 1, wherein the film coating having said first color is applied to said oral solid dosage form until a weight gain of from about 0.25 to about 15% by weight is achieved.

18. The method of claim 17, wherein the film coating having said first color is applied to said oral solid dosage form until a weight gain of from about 1 to about 10% by weight is achieved.

19. The method of claim 1, wherein the first color found in the film coating applied to the oral solid dosage form is darker than the second color in the ink applied to the oral solid dosage form after film coating.

20. The method of claim 1, wherein the first color found in the film coating applied to the oral solid dosage form is lighter than the second color in the ink applied to the oral solid dosage form after film coating.

21. A method of applying an identification mark on a pharmaceutical dosage form, comprising:
a) providing an oral solid dosage form;
b) applying a film coating containing a pearlescent pigment therein and having a first color to said oral solid dosage form; and
c) applying an identification mark to the film coated oral solid dosage form prepared as a result of step b), using an ink having a second color which is substantially similar in color to said first color.

22. An oral solid dosage form prepared by the process of claim 1.

23. An oral solid dosage form prepared by the process of claim 21.
